(19) United States
(12) Patent Application Publication
(10) Pub. No.: US 2006/0128002 A1
(43) Pub. Date: Jun. 15, 2006

(54) Method for supplying a biologically utilized aqueous system with oxygen, and device and set for carrying out said method

(76) Inventor: Klaus Sochting, Attenham (DE)

Correspondence Address: MONAHAN & MOSES, LLC, 13-B W. WASHINGTON ST., GREENVILLE, SC, 29601, US

(21) Appl. No.: 10/559,045

(22) PCT Filed: May 28, 2004
(86) PCT No.: PCT/EP04/05791

§ 371 Date: Feb. 8, 2006

(30) Foreign Application Priority Data
May 30, 2003 (DE) 103 24 563.4

(51) Int. Cl. C12P 3/00; C12M 1/04

(52) U.S. Cl. 435/168; 435/818; 435/304.2; 210/606

(57) ABSTRACT

The invention relates to a method for supplying a biologically utilized aqueous system with oxygen, comprising the following steps: i) a predetermined quantity of catalase is added to the biologically utilized aqueous system; ii) H₂O₂ or a salt thereof is added to the biologically utilized aqueous system; and iii) the peroxide added to the system is catalytically decomposed under the effect of the catalase in water and O₂. Preferably, step ii) is carried out in a continuous manner by discharging an aqueous peroxide solution from a storage container into the biologically utilized aqueous system by means of a gas pressure-generating chemical reaction that takes place in the storage container. The invention further relates to a device and a set for carrying out said preferred method.

The present invention relates to a method for supplying a biologically utilised system with oxygen, that is, the generation of oxygen in a biologically utilised aqueous system, particularly in a system having an elevated oxygen requirement, and a device and a set for carrying out said method.

Aerobic (oxygen-consuming) organisms whose natural environment is aqueous systems, draw the oxygen (O₂) necessary to their metabolism from the water surrounding them in which the oxygen is present in dissolved form. The oxygen taken up by the organisms is converted in metabolic processes into CO₂. Replacement of oxygen into the aqueous system takes place, on the one hand, by diffusive dissolution processes at the boundary surface between the aqueous system and the atmosphere, and by photosynthesising organisms present in the aqueous system. Since the processes feeding oxygen into the aqueous system take place relatively slowly, the possible occupation density of oxygen-consuming organisms in the aqueous solution is restricted.

However, situations exist in which a higher level of occupation (a high organism density with crowded cultivation) with oxygen-consuming organisms is desired than the oxygen supply by natural means would allow. In such a case, oxygen must be fed in.

Examples of crowded cultivation of this type are the keeping of fish or other aquatic animals for ornamental purposes or as food, or during transport, or for short-term display, for example, in an ornamental fish exchange, or for temporary storage during maintenance and/or cleaning operations on aquaria, ponds, breeding containers, etc.

The commonest method for supplying additional oxygen into an aqueous system is finely distributed injection of air or pure oxygen with suitable technical equipment. However, a large part of the gas escapes from the aqueous system, so that this method can only be carried out in open tanks. Furthermore, a substantial technical effort is required. Electrically operated feed apparatus including the required power supply must be provided, together with technical equipment allowing the use of pressurised gas tanks. In many fields, this effort is not possible and/or desired, examples being in the cleaning of fishponds and aquaria, the transport of fish, for example, of ornamental fish, bait fish or stock fish and the display of ornamental fish at a fish exchange.

The quantity of oxygen dissolved in the water is less than 10 mg/l under normal environmental conditions. A fish needs about 1 mg oxygen per g of body weight per hour. Furthermore, for efficient uptake of oxygen from the water, a certain minimum concentration must be present. Therefore, the oxygen quantity stored in the water without external supply is sufficient only for a very small occupation density and keeping duration.

For greater occupation densities and/or longer keeping durations, the additional supply of oxygen is essential.

If the technically complex method described above is to be avoided, only a few alternative possibilities are available.

In closed systems, such as a fish transport bag, a gas bubble (air, oxygen, or mixtures thereof is included above the water surface. However this gas bubble occupies a large volume (in practice the ratio of water: gas bubble is approximately 1:5), and the oxygen must be washed into the water through movement of the bag.

Another method for supplying a biologically utilised aqueous system with oxygen is disclosed in DE 31 090 64 and the parallel U.S. Pat. No. 4,466,556. Therein an H₂O₂ solution is slowly and continuously decomposed in a separate container by means of a ceramic catalyst and thereby discharged through an outlet opening. A further solid catalyst surrounding the outlet opening ensures that the H₂O₂ from the discharged solution is decomposed and oxygen is thereby released. Since the solid catalyst is locally restricted, the oxygen is also only produced locally to its surface.

It is therefore an object of the present application to provide a method which enables the supply of oxygen into an aqueous system with a high occupation density of oxygen-consuming organisms to be assured over a long period.

According to the invention, this aim is achieved with a method which enables the supply of a biologically utilised aqueous system with oxygen, as defined in claim 1. Preferably, supply with oxygen takes place in a continuous manner, that is, for a previously determinable time substantially constantly, as defined in claim 2. Further preferred embodiments are given by the claims 3 to 10.

Furthermore, the above aim is fulfilled by the device according to the invention for carrying out the method according to the invention, as defined in claim 11. Preferred embodiments are contained in claims 12 to 18.

Also suitable for achieving the above aim is the set provided in accordance with the invention according to claim 19, which comprises a device according to the invention and the starting substances necessary to carrying out the method.

The method according to the invention for continuous supply of a biologically utilised aqueous system with oxygen comprises the following steps:
i) a predetermined quantity of a catalase is added to the biologically utilised aqueous system,
ii) H₂O₂ or a salt thereof is added to the biologically utilised aqueous system, and
iii) the peroxide added to the system is catalytically decomposed, under the effect of the catalase, to water and O₂.

Preferably, step ii) is carried out by discharging an aqueous peroxide solution from a storage container into the biologically utilised aqueous system by means of a gas pressure-generating chemical reaction taking place in the storage container.

With this method, the device according to the invention and the set used for carrying out the method, the following advantageous effects are achieved:
The release of O₂ is carried out in a space-saving manner and economically in a quantity per unit time that is suitable for high occupation densities.
A large quantity of O₂ can be stored in the starting substances and released over a long period.
The O₂ quantity generated per unit time can be adjusted according to need and (at constant temperature) kept constant over a long period.
In the preferred embodiment according to claim 2, a further particular advantage is that the oxygen demand of fish or other organisms associated with changes in temperature is compensated for by the automatically adjusted oxygen release. Adjustment of the oxygen release based on temperature variations is therefore not necessary.
Through the use of a catalyst (catalase) which itself is finely distributed in the aqueous system, the oxygen is generated in the entire volume, that is, severe concentration differences or even local undersupply do not occur, even given a high occupation density of oxygen-consuming organisms in the aqueous system.
In aquaria or in transport or intermediate containers, any desired quantity of O₂ per unit time can be provided in simple manner without technically complex apparatus, hose connections and/or cabling being necessary.

The peroxide solution mentioned above is preferably a solution of hydrogen peroxide (H₂O₂) in water. Other peroxide solutions may be used such as, for example, solutions of salts (e.g. addition salts) of H₂O₂, which in aqueous solution exist in equilibrium with free H₂O₂. The concentration of the peroxide is preferably in the range of 5% to 50% by weight, more preferably in the range of 10% to 30% by weight and still more preferably in the range of 15% to 25% by weight.

It is noteworthy that the method according to the invention may also be carried out outside the concentration range given above. A concentration of less than 5% by weight of peroxide (preferably H₂O₂) is impracticable, however, since with falling concentration, the ratio of the volume of peroxide solution to the total quantity of oxygen releasable therefrom becomes ever greater.

Peroxide solutions, particularly H₂O₂ solutions, having a concentration of more than 50% by weight of H₂O₂ have a very good ratio of peroxide solution to oxygen quantity releasable therefrom, although solutions of this type become increasingly unstable with rising peroxide concentration and can be inclined to decompose. Therefore their handling by inexperienced users is not without risk.

The gas pressure-producing reaction preferred according to the invention used for discharging the peroxide solution out of the storage container is any reaction that leads in controlled manner to the generation of a gaseous product. Preferably, it is the conversion of a small quantity of the peroxide present in the aqueous peroxide solution, releasing oxygen as a gaseous reaction product.

The generation of the gaseous reaction product can be catalysed with a suitable catalyst. With suitable dosing of the catalyst, the quantity of gas generated per unit time and thus the discharge rate of the peroxide solution can be precisely adjusted within a broad range. For this purpose, a chronological variation in temperature need not be considered, since the variation in oxygen demand of fish and other organisms associated with temperature changes is compensated for by the automatically adjusted oxygen release.

Suitable for use as catalysts are any of the compounds known from the prior art as decomposition catalysts. If the decomposition of a small proportion of the H₂O₂ present in the peroxide solution is used as a gas pressure-generating reaction, any catalyst which brings about the decomposition of H₂O₂ into H₂O and gaseous O₂ may be used. MnO₂ or platinum are preferred catalyst materials, and particularly preferred is ceramically bound MnO₂. Mixtures of two or more mutually compatible catalysts may also be used.

The rate at which gas production (for example, the decomposition of H₂O₂) takes place in the storage container is determined by the quantity and type of catalyst and the reaction temperature, that is, the temperature of the ambient aqueous system.

The peroxide solution is discharged from the storage container by the gas pressure-producing reaction through one or a plurality of fine opening(s) from the storage container and is absorbed by the surrounding aqueous system. The fact that the peroxide solution itself and not the gas generated in the storage container emerges through the opening(s) is ensured by a suitable design of the device for carrying out the method according to the invention. The opening(s) are also preferably designed so that penetration of the surrounding aqueous medium into the storage container is prevented, for example, by design of the outlet opening in the form of a diving bell. The aspects relating to the device for carrying out the method according to the invention will be described in greater detail later.

The catalase used for release of oxygen from the peroxide solution in aqueous solution can be any catalase. Examples are catalases gathered from animal liver, in particular beef liver, blood or microorganisms. Preferred are catalases taken from microorganisms, since they have a greater degree of stability than other catalases.

The catalase is added to the aqueous solution in a quantity such that it suffices to decompose the supply of peroxide provided. In order to decompose the peroxide contained in 1 ml of a 19.9% H₂O₂ solution, 2000 units of catalase are required.

The device for carrying out the method according to the invention will now be described.

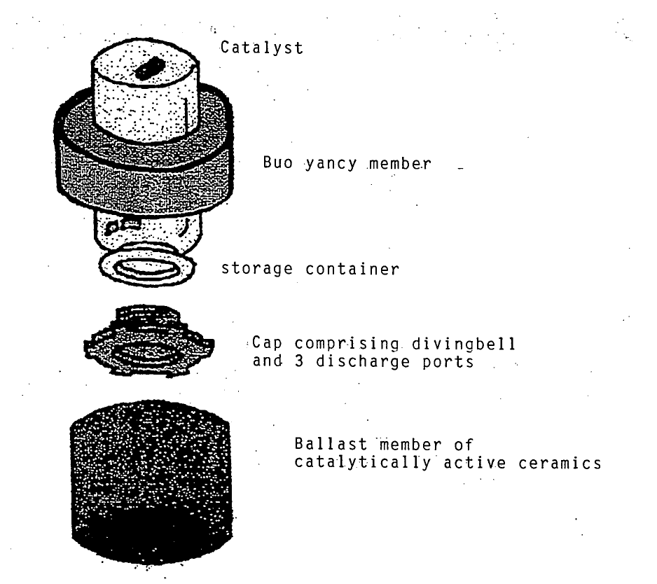

An exemplary and preferred embodiment of the device according to the invention is shown schematically in FIG. 1.

The device comprises a storage container (1) with at least one outlet opening, which permits the discharge of the aqueous peroxide solution due to the gas pressure-generating reaction taking place in the storage container, and a device (2) which ensures that, due to the gas pressure-generating reaction, the peroxide solution and not the gas generated emerges from the at least one outlet opening.

The at least one outlet opening is dimensioned so that the peroxide solution cannot flow out through the outlet opening under the relevant conditions. On the other hand, the at least one opening is large enough so that emergence of a drip due to the pressure built up through the catalysed decomposition process taking place in the storage container is possible. Preferably, the at least one outlet opening has a circular or almost circular cross-section with a diameter of ≦1 mm, more preferably ≦0.5 mm, and yet more preferably ≦0.1 mm. When used during transport, it must be ensured that the acceleration of the solution brought about by blows does not lead to it running out of the opening. Therefore diameters of ≦0.1 mm are preferred for such applications.

In a preferred embodiment, the storage container (1) is designed in two parts and comprises a container (1a) and a stopper (1b) placeable firmly and in watertight manner thereon. The container (1a) may be made from any desired transparent or opaque material, preferably glass or plastics. The stopper (1b) placeable firmly and in watertight manner on the container is preferably made of a plastics material and is preferably a pressed-in stopper or screw closure.

Generally not preferred for the container (1a) are metals, since these are able to catalyse the decomposition of the peroxide solution. It may be that a catalysing effect of this type is desirable in principle, but due to the falling level in the storage container during the method according to the invention, the speed of this decomposition reaction is not linear. As a result, the rate at which the peroxide solution is driven out of the storage container would become slower with increasing duration of the method according to the invention. Insofar as such an effect is desired, however, a container (1a) made of metal may be used.

Preferably the stopper (1b) is made so that it has the form of a diving bell so that no water can penetrate from outside into the storage container.

The device (2) preferably comprises a ballast member (2a) and a buoyancy member (2b), which are linked to the storage container (1) in a manner such that the at least one outlet opening is always downwardly directed during carrying out of the method according to the invention.

The ballast member (2a) is a weight which during operation of the device according to the invention ensures that the at least one outlet opening of the storage container (1) is always downwardly directed. The buoyancy member (2b) prevents tipping over.

According to a particularly preferred embodiment according to the invention, the stopper (1b) is provided with the at least one discharge opening and the ballast member is a ring or bowl-shaped element into which the stopper is firmly inserted.

Preferably the ballast member (2a) is made of a material which is catalytically active with regard to the decomposition of the peroxide, releasing O₂. An example of a material of this type is ceramically bound MnO₂. The buoyancy member (2b) is made of a light material which is able to float, for example, cork, expanded polystyrene, foam material, etc.

The set according to the invention for carrying out the method according to the invention comprises the device according to the invention and described above and a standard quantity of an aqueous peroxide solution, preferably a hydrogen peroxide (H₂O₂) solution, a catalyst for catalysing the gas pressure-generating chemical reaction and a standard quantity of an aqueous catalase solution.

The preferred concentrations of peroxide solution and the preferred catalysts and catalases have been described above in connection with the method according to the invention.

The carrying out of the method according to the invention will now be described by reference to the preferred embodiments of the device according to the invention described above.

The container (1a) of the storage container (1) is fed with a standard quantity of an aqueous peroxide solution and the catalyst for the gas pressure-generating reaction taking place in the storage container and firmly closed with the stopper (1b). Then the ballast member (2a) and the buoyancy member (2b) are fixed to the storage container (1) and this arrangement is placed in the aqueous system to be supplied with oxygen, for example, a bucket or a plastic bag with fish. Furthermore, a particular quantity of catalase is dripped into the aqueous medium as described above.

As a result of the gas pressure-generating reaction in the storage container, the peroxide solution therein is discharged from the storage container. H₂O₂ (directly in the free form from the peroxide solution or from the equilibrium described above between salt and free H₂O₂) is then decomposed in the aqueous system by the added catalase to H₂O and O₂. The O₂ produced dissolves in the aqueous system and is available to the oxygen-consuming organisms, which are present in the aqueous system, for their metabolic processes.

The present invention will now be further described by reference to an example, which has a purely representative character and should not be regarded as the exclusive subject matter of the invention.

According to an exemplary embodiment, the storage container (1) comprises a glass container, for instance with a volume of 30 ml, as the container (1a) and a plastic stopper (1b), which is provided with a plurality of outlet openings. The glass container is filled up to a predetermined fill level with, for example, a solution of 19.9% by weight of H₂O₂ and closed with the stopper. Subsequently, a ceramic bowl with a catalytic effect with regard to the decomposition of H₂O₂ and functioning as a ballast member (2a) is placed on this stopper. With the aid of a flotation ring (2b), the arrangement is brought into equilibrium and placed into the aqueous system to be supplied with oxygen, to which 7 drops (⅓ ml) of an aqueous catalase solution with a catalase concentration of 170,000 U/ml have been added (corresponding to approximately 2,000 U per ml of H₂O₂ solution).

The arrangement described above is suitable for supplying an aqueous system, for example, an open container for fish transport having a water volume of approximately 2-20 l. At a temperature of 9°, the operational duration is approximately 144 hours and the rate of O₂ release is approximately 22 mg/h. An increase in the water temperature by 8° C. causes an approximate doubling of the rate of decomposition of peroxide in the storage container and consequently its discharge rate. With conditions otherwise the same, at 17° C., the operational duration is approximately 72 hours and the O₂ quantity released per hour is approximately 44 mg/h. Accordingly, at a temperature of 25° C., the operating duration is approximately 36 hours, and the rate of O₂ release is approximately 88 mg/h.

Given the details set out here, it is possible for a person skilled in the art without further difficulty to adjust the relevant operating parameters for all conceivable conditions, that is, for different water temperatures, different occupation densities of the aqueous system with oxygen-consuming organisms, different desired operational durations of the method according to the invention, etc. This includes the proper selection of the type and quantity of the catalyst, the quantity of peroxide solution and the quantity of catalase to be fed in. The details given above are therefore transferable in simple manner to a plurality of different conditions and requirements.

1. Method for supplying a biologically utilised aqueous system with oxygen, comprising the following steps:
i) addition of a predetermined quantity of a catalase to the biologically utilised aqueous system,
ii) addition of H₂O₂ or a salt thereof to the biologically utilised aqueous system, and
iii) catalytic decomposition of the peroxide added to the system under the action of the catalase to water and O₂.

2. Method according to claim 1, wherein step ii) is carried out by discharging an aqueous peroxide solution from a storage container into the biologically utilised aqueous system by means of a gas pressure-generating chemical reaction taking place in the storage container.

3. Method according to claim 1, wherein the aqueous peroxide solution is a hydrogen peroxide (H₂O₂) solution.

4. Method according to claim 1, wherein the biologically utilised aqueous system is a system which has an elevated oxygen requirement, due to a high occupation density of oxygen-consuming organisms.

5. Method according to claim 4, where in the oxygen-consuming organisms are fish.

6. Method according to claim 2 wherein the gas pressure-generating chemical reaction in the storage container is the decomposition of hydrogen peroxide with release of oxygen.

7. Method according to claim 2, wherein the gas pressure-generating chemical reaction is catalysed by one or more catalyst(s).

8. Method according to claim 7, wherein the catalyst is manganese dioxide.

9. Method according to claim 8, wherein the manganese dioxide is ceramically bound.

10. Method according to claim 1, wherein the catalase is a catalase extracted from microorganisms.

11. Device for carrying out the method according to claim 2, comprising
i) a storage container (1) with at least one outlet opening which permits the emergence of the aqueous peroxide solution into the biologically utilized medium due to the gas pressure-generating reaction taking place in the storage container,
ii) a device (2) which provides that due to the gas pressure-generating reaction, the peroxide solution, but not the gas generated, emerges from the at least one outlet opening, wherein the device (2) comprises a ballast member (2a) and a buoyancy member (2b), which are linked to the storage container (1) in a manner such that, during carrying out the method, the at least one outlet opening is downwardly directed.

12. Device according to claim 11, wherein the storage container (1) is constructed in two parts and comprises a container (1a) and a stopper (1b) placeable thereon firmly and in a waterproof manner.

13. Device according to claim 12, wherein the stopper (1b) is provided with the at least one outlet opening, which allows the emergence of the aqueous peroxide solution due to the gas pressure-generating reaction occurring in the storage container.

14. Device according to claim 13, wherein the stopper (1b) is designed in the form of a diving bell.

15. (canceled)

16. Device according to claim 12 wherein the ballast member (2a) is a weight to be attached to the stopper (1b) and/or the container (1a) of the storage container.

17. Device according to claim 16, wherein the ballast member (2a) comprises catalytically active material capable of decomposing H₂O₂, releasing O₂.

18. Device according to claim 16 wherein the buoyancy member (2b) is a flotation ring to be attached to the storage container at a variable height.

19. Set for carrying out the method according to claim 2, comprising
a device comprising
a) a storage container (1) with at least one outlet opening which permits the emergence of the aqueous peroxide solution due to the gas pressure-generating reaction taking place in the storage container, and
b) a devive (2) which provides that due to the gas pressure-generating reaction, the peroxide solution, but not the gas generated, emerges from the at least one outlet opening,
ii) a standard quantity of an aqueous hydrogen peroxide (H₂O₂) solution,
iii) at least one catalyst catalysing the gas pressure-generating chemical reaction in the storage container, and
iv) a standard quantity of an aqueous catalase solution.

20. Set of claim 19, wherein the storage container (1) is constructed in two parts and comprises a container (1a), and a stopper (1b) containing the at least one outlet opening.

21. Set of claim 19, wherein the device (2) comprises a ballast member (2a) and a buoyancy member (2b), which are linked to the storage container (1) in a manner such that, during carrying out the method, the at least one outlet opening is downwardly directed.
